(19) United States
(12) Patent Application Publication
(10) Pub. No.: US 2026/0124275 A1
(43) Pub. Date: May 7, 2026

(54) METHODS OF TREATING DIABETES AND RELATED COMPLICATIONS

(71) Applicant: THE HONG KONG POLYTECHNIC UNIVERSITY, Hong Kong (CN)

(72) Inventors: Chi Ming WONG, Hong Kong (CN); Ka Ying CHAN, Hong Kong (CN)

(21) Appl. No.: 18/939,610

(22) Filed: Nov. 7, 2024

(51) Int. Cl. A61K 38/18; A61P 3/10

(52) U.S. Cl. CPC A61K 38/1808; A61P 3/10

(57) ABSTRACT

A method of treating diabetes or a related complication in a subject in need thereof, the method comprising: administering a therapeutically effective amount of epithelial mitogen or a functional fragment thereof to the subject. The method can be used to lower the amount of circulating glucose in the subject by enhancing glucose uptake by hepatocytes and myocytes in an insulin-dependent and independent manner, and pancreatic beta cell regeneration.

### REFERENCE TO SEQUENCE DISCLOSURE

The sequence listing identified as “Sequence_Listing_P26296US00” in XML file format having a file size of 8.0 KB created on Oct. 16, 2024, filed herewith, is incorporated herein by reference in its entirety.

### TECHNICAL FIELD

The present disclosure relates to methods for treating diabetes and related complications.

### BACKGROUND

Diabetes is a growing global health concern, with increasing prevalence in both wealthy and relatively more developed countries. The primary underlying causes are impaired insulin production and reduced insulin utilization effectiveness. Over time, the elevated glucose levels characteristic of diabetes can lead to a range of serious complications, including heart attack, stroke, blindness, kidney failure, and lower limb amputation. These complications place a significant burden on healthcare systems and society as a whole. Unfortunately, there is currently no complete cure for diabetes. Patients with mild to severe diabetes must typically rely on medications to help maintain their blood glucose levels within a healthy range. Diabetes drugs can be categorized by their mechanisms of action and/or routes of administration. Protein-based therapeutics, such as glucagon-like peptide (GLP-1) agonists, have emerged as a promising class of diabetic treatments. These agents tend to have more targeted therapeutic actions, lower side effect profiles, and are generally better tolerated compared to some traditional small-molecule drug options. Two examples are Liraglutide (marketed as Victoza) and Exenatide (marketed as Byetta), both of which have been used clinically for the management of diabetes.

The use of insulin secretion stimulants, while effective for some types of diabetes, still has some concerning associated issues. Studies have suggested that primary insulin hypersecretion can actually lead to beta cell dysfunction, as seen in patients with persistent hyperinsulinemic hypoglycemia of infancy (PHHI) who have genetic mutations causing excessive insulin release. In fact, a recent study indicated that an autosomal dominant mutation in the sulfonylurea receptor-1 (SUR1) causing congenital hyperinsulinism can subsequently result in insulin-deficient diabetes, representing a new genetic subclass of type 2 diabetes with similar characteristics of glucose intolerance, beta cell dysfunction, and hyperglycemia. In vitro studies have also suggested that chronic treatment of beta cells with insulin secretagogues, such as sulfonylureas, can induce cell death through apoptosis. Conversely, allowing beta cells to “rest” by reducing insulin secretion could be beneficial and protect against glucose toxicity and oxidative stress. This helps explain the high failure rate of current first- and second-line diabetes therapies, where up to 15-34% of patients fail at 5 years. Alternative approaches to treating diabetes that do not solely rely on stimulating insulin secretion are therefore needed. In contrast to type 2 diabetes, type 1 diabetes is an autoimmune disease that destroys insulin-producing beta cells, rendering patients unable to naturally regulate their blood sugar. As a result, agents that stimulate insulin secretion cannot be used to treat type 1 diabetes. Management of type 1 diabetes is challenging, requiring constant monitoring of blood sugar and insulin administration via injections or pump. Even with careful management, type 1 diabetes patients remain at significant risk of long-term complications. Finding effective treatments to improve quality of life for these patients is a crucial priority.

Therefore, there is an unmet need for an effective drug for both type 1 and type 2 diabetes that eliminates or at least diminishes the disadvantages and problems described above.

The epidermal growth factor receptor (EGFR) and its ligands have been implicated in the pathogenesis of diabetes. Studies have shown that EGFR and its ligands, such as epidermal growth factor (EGF) and transforming growth factor-alpha (TGF-α), play a role in insulin resistance, pancreatic beta-cell function, and glucose homeostasis. Dysregulation of the EGFR signaling pathway has been associated with the development and progression of both type 1 and type 2 diabetes. Therefore, EGFR ligands have great potential in the development of new therapeutic strategies targeting this pathway.

There is thus a need for improved methods for treating diabetes that address or overcome at least some of the disadvantages described above.

### SUMMARY

Accordingly, in a first aspect of the present disclosure provides a method of treating diabetes or a related complication in a subject in need thereof, the method comprising: administering a therapeutically effective amount of epithelial mitogen (Epigen) or a functional fragment thereof to the subject.

In certain embodiments, the diabetes is type I diabetes, type II diabetes, type I pre-diabetes, or type II pre-diabetes.

In certain embodiments, the related complication is selected from the group consisting of glucose intolerance, hyperglycemia, insulin resistance, reducing fat mass, pancreatic beta cell dysfunction, hypertension, cardiovascular diseases comprising hypertension, heart attack, and stroke, diabetic nephropathy, diabetic retinopathy, diabetic vasculopathy, diabetic neuropathy, blindness, kidney failure, diabetic foot and lower limb amputation and combinations thereof.

In certain embodiments, the Epigen comprises a polypeptide having at least 95% sequence homology with SEQ ID NO: 1 or SEQ ID NO: 2.

In certain embodiments, the Epigen comprises a polypeptide having at least 98% sequence homology with SEQ ID NO: SEQ ID NO: 1 or SEQ ID NO: 2.

In certain embodiments, the Epigen comprises a polypeptide having at least 99% sequence homology with SEQ ID NO: SEQ ID NO: 1 or SEQ ID NO: 2.

In certain embodiments, the Epigen comprises a polypeptide having SEQ ID NO: SEQ ID NO: 1 or SEQ ID NO:2.

In certain embodiments, the Epigen consists of a polypeptide having SEQ ID NO: SEQ ID NO: 1 or SEQ ID NO:2.

In certain embodiments, the Epigen is a recombinant Epigen.

In certain embodiments, the Epigen is in isolated form.

In certain embodiments, the diabetes is type I diabetes or type II diabetes and the consists of a polypeptide having SEQ ID NO: 1 or SEQ ID NO: 2.

In certain embodiments, the Epigen is administered intravenously, intraperitoneally, or intramuscularly, orally, or subcutaneously.

In certain embodiments, the subject is a human.

This summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. Other aspects of the present invention are disclosed as illustrated by the embodiments hereinafter.

### BRIEF DESCRIPTION OF DRAWINGS

The appended drawings, where like reference numerals refer to identical or functionally similar elements, contain figures of certain embodiments to further illustrate and clarify the above and other aspects, advantages and features of the present invention. It will be appreciated that these drawings depict embodiments of the invention and are not intended to limit its scope. The invention will be described and explained with additional specificity and detail through the use of the accompanying drawings in which:

FIG. 1. Epigen protein sequence alignment. Protein sequence alignment between human (hEPGN, SEQ ID: 1) and mouse epigen (mEPGN SEQ ID: 2), in the absence of their respective signal peptide, showed 77.3% identity and 83.9% similarity.

FIG. 2. Circulating Epigen level was reduced in obese mice and fasting state. (A) mRNA level of EPGN in various tissue. (B) Protein expression of Epigen protein in various tissue. (C) Epigen level in feeding, 16 hr fast and 2 hr refed of mice fed with STC or HFD for 6 weeks. (D) Corresponding glucose level.

FIG. 3. EPGN Knockout (KO) mice exhibit impaired glucose homeostasis on HFD feeding. (A) Schematic diagram of experiment. (B) Schematic diagram of EPGN knockout and genotyping strategy. (C) Genotyping. (D) EPGN mRNA level of tongue. (E) Circulating level of Epigen. (F) Body weight. (G) Fasting glucose. (H) Impaired body composition on 16 W HFD feeding. (I) Adipose tissue weight. (J) H&E staining of sWAT and eWAT. (K) Quantification of adipocytes size in sWAT and eWAT. (L) Quantification of immune cell infiltration in sWAT. GTT on 14 W HFD feeding (M), AUC (N), % of basal (O) and GSIS (P). ITT on 15 W HFD feeding (Q), AUC (R), % of basal (S). PTT on 16 W HFD feeding (T), AUC (U), % of basal (V).

FIG. 4. Global EPGN deletion exacerbates obesity and nonalcoholic fatty liver disease. (A) Triglyceride level. (B) Total cholesterol level. (C) LDL and HDL level. (D) Free fatty acid (FFA) level. Hepatic TG (E) and CHO (F) level in liver (6 months HFD feeding). Quantification (G) and representative H&E staining images (H) of liver (6 months HFD feeding). (I) AST and ALT level in serum (6 months HFD feeding).

FIG. 5. HFD mice overexpressing EPGN with adenovirus exhibit improved glucose homeostasis. (A) Schematic diagram of experiment, Adv-GFP or Adv EPGN were injected into mice fed with HFD for 12 weeks. (B) Relative EPGN mRNA level in liver after sacrifice. (C) Epigen level increased in liver of adv-EPGN group. (D) Epigen level in serum at day 3. (E) Body weight. (F) Fasting glucose. (G) Improved glucose tolerance of HFD mice overexpressing EPGN, AUC (H) and GSIS (I). PTT (J) and corresponding AUC (K). ITT (L) and AUC (M).

FIG. 6. Acute treatment of recombinant Epigen alleviates glucose intolerance in mice and induces GSIS. GTT (A), AUC (B) GSIS (C) on 18-week HFD-fed mice. GTT (D), AUC (E) GSIS (F) on STZ-treated mice. (G) IVIS image of label-Epigen protein distribution. (H) IVIS image of NBDG distribution. (I) Epigen induced insulin secretion of R cells in the present of high glucose stimulation. (J) Epigen induced insulin secretion in R cells and was blocked by EGF receptor inhibitor AG1478. (K) Epigen induced insulin secretion was blocked by calcium channel inhibitor Nifedipine (Nif). (L) Epigen induced glucose uptake in 3T3-L1 differentiated adipocytes in a dose-dependent manner. (M) The effect of Epigen can be blocked by EGF receptor inhibitors. (N,O) Insulin was used as a positive control for glucose uptake in 3T3-L1. (P) Epigen did not induce glucose uptake in HepG2 cells. (Q) Glucagon and pyruvate can induce glucose production of primary hepatocytes. (R) Epigen inhibited glucose production of primary hepatocytes under glucagon and pyruvate induction. (S) Overexpression of EPGN via adenovirus inhibited gluconeogenesis in primary hepatocytes. (T) Epigen induced glucose uptake into L6 differentiated muscle cells in a dose-dependent manner. (U) The effect of Epigen can be blocked by EGF receptor inhibitors AG1478 and Tucatinib in L6 cells.

FIG. 7. Daily Epigen injection reduced fat mass and improved glucose homeostasis in HFD-fed obese mice. (A) Schematic diagram of experiment, recombinant Epigen protein were IP injected 2 mg/kg daily into mice fed with HFD for 20 weeks, and injection of for 15 weeks. (B) Body weight. (C) Body composition. (D) Weight of sWAT and eWAT. (E) Quantification of adipocytes size of sWAT and eWAT. (F) Representative images of H&E staining of sWAT and eWAT. (G) Quantification of immune cell infiltration in sWAT. (H) Fast glucose. GTT (I), AUC (J), % of basal (K) and GSIS (L) on 10 W Epigen injection. PTT (M), AUC (N) and % of basal (O) on 14 W injection. ITT (P) and AUC (Q) on 12 W injection.

FIG. 8. Daily Epigen injection improved glucose homeostasis in STZ-induced diabetic mice. (A) Schematic diagram of experiment. (B) Body weight. (C) Fasting glucose. (D) Change of fast insulin level. GTT (E), AUC (F), % of change (G) and GSIS (H) STZ mice after 6 weeks of Epigen treatment. ITT (I) and AUC (J) after 7 weeks of Epigen treatment. (K) Pancreas weight. (L) Total amount of insulin in pancreas. (M) Quantification of BrdU+/insulin+ cells. (N) Representative images of BrdU staining of pancreases.

FIG. 9. Epigen knockout (KO) mice did not alter glucose homeostasis on STC feeding. (A) Schematic diagram of experiment. (B) EPGN mRNA level of tongue. (C) Fasting glucose. (D) Body weight. (E) Body composition on 15 W. GTT (F) and AUC (G) on 14 W. ITT (H) and AUC (I) on 15 W. PTT (J) and AUC (K) on 16 W.

FIG. 10. Slightly reduced energy expenditure in HFD-feeding EPGN-KO mice (3 months HFD-feeding). Real-time monitoring curve of oxygen consumption (A), carbon dioxide release (B), energy consumption (C) and respiratory exchange ratio (D) of STC-feeding 16 W old EPGN-KO mice and their WT littermates. Quantification of activity (E) and accumulated food intake (F) of STC-feeding 16 W old EPGN-KO mice and their WT littermates. Real-time monitoring curve of oxygen consumption (G), carbon dioxide release (H), energy consumption (I) and respiratory exchange ratio (J) of STC-feeding 16 W old EPGN-KO mice. Quantification of activity (K) and accumulated food intake (L) of EPGN-KO mice and their WT littermates after 12 weeks HFD feeding.

FIG. 11. Chronic Epigen protein injection increased energy expenditure and improved lipid profile. (A) Real-time monitoring curve and average oxygen consumption (VO₂). (B) Real-time monitoring curve and averaged of carbon dioxide release (VCO₂). (C) Real-time monitoring curve and average energy consumption. (D) Real-time monitoring curve and average Respiratory exchange ratio (RER=VCO₂/VO₂). (E) Quantification of activity. (F) Quantification of accumulated food intake. (G) TG and CHO level. (H) LDL and HDL level. (I) FFA level. (J) Hepatic lipid level. Representative images of liver H&E staining (K) and quantification of steatosis (L). Representative images of liver Sirius Red staining (M) and quantification of fibrosis (N) in liver. (O) AST and ALT level.

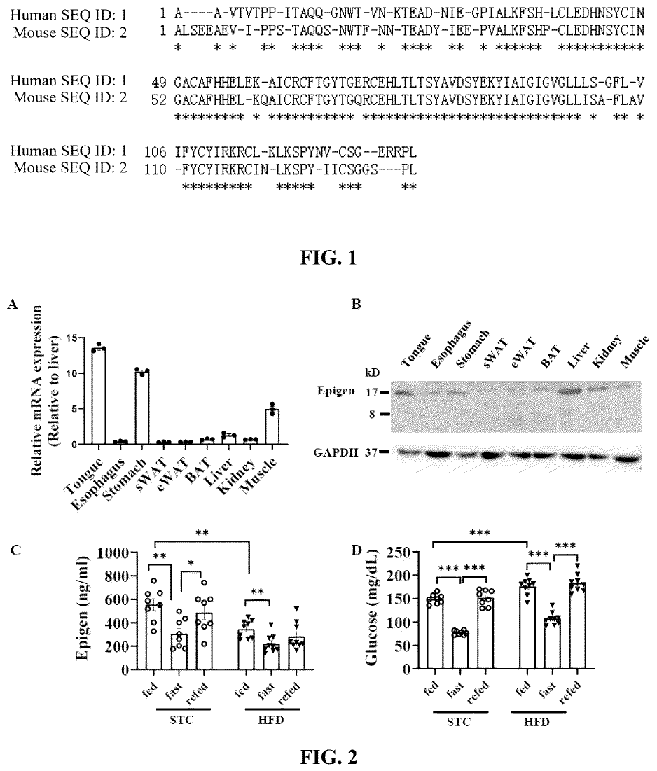

Skilled artisans will appreciate that elements in the figures are illustrated for simplicity and clarity and have not necessarily been depicted to scale.

### DETAILED DESCRIPTION

### Definitions

The definitions of terms used herein are meant to incorporate the present state-of-the-art definitions recognized for each term in the field of biotechnology. Where appropriate, exemplification is provided. The definitions apply to the terms as they are used throughout this specification, unless otherwise limited in specific instances, either individually or as part of a larger group.

The term “protein” or “polypeptide” as used herein indicates an organic polymer composed of two or more amino acid monomers and/or analogs thereof. The term “polypeptide” includes amino acid polymers of any length including full length proteins and peptides, as well as analogs and fragments thereof. A polypeptide of three or more amino acids is also called an oligopeptide. As used herein, the term “amino acid”, “amino acidic monomer”, or “amino acid residue” refers to any of the twenty naturally occurring amino acids including synthetic amino acids with unnatural side chains and including both D and L optical isomers. The term “amino acid analog” refers to an amino acid in which one or more individual atoms have been replaced, either with a different atom, isotope, or with a different functional group but is otherwise identical to its natural amino acid analog.

As used herein, the term “unnatural amino acid” refers to any amino acid, modified amino acid, and/or amino acid analogue that is not one of the 20 common naturally occurring amino acids, seleno cysteine or pyrrolysine.

As used herein, the term “variant” refers to a polynucleotide or nucleic acid differing from a reference nucleic acid or polypeptide, but retaining essential properties thereof. Generally, variants are overall closely similar, and, in many regions, identical to the reference nucleic acid or polypeptide.

A variant can, for example, comprise the amino acid sequence of the parent polypeptide sequence with at least one conservative amino acid substitution. Alternatively or additionally, the variant can comprise the amino acid sequence of the parent polypeptide sequence with at least one non-conservative amino acid substitution. In this case, it is preferable for the non-conservative amino acid substitution to not interfere with or inhibit the biological activity of the functional variant. The non-conservative amino acid substitution may enhance the biological activity of the variant, such that the biological activity of the variant is increased as compared to the parent polypeptide.

The term “functional fragment” when used in reference to a polypeptide refers to any part or portion of the subject polypeptide, which part or portion retains the biological activity of the polypeptide of which it is a part (the parent polypeptide). The functional fragment can be any fragment comprising contiguous amino acids of the polypeptide of which it is a part, provided that the functional fragment still exhibits at least 40%, 50%, 60%, 70%, 80%, 90%, 95%, or 99% or has substantially the same or even higher biological activity of the parent polypeptide. In reference to the parent polypeptide, the functional fragment can comprise, for instance, about 10%, 20%, 25%, 30%, 40%, 50%, 60%, 70%, 80%, 90%, 95%, 98% or more, of the parent polypeptide.

The functional fragment can comprise additional amino acids at the amino or carboxy terminus, or at both termini, e.g., amino acids not found in the amino acid sequence of the parent polypeptide.

Amino acid substitutions of the described polypeptides can be conservative amino acid substitutions. Conservative amino acid substitutions are known in the art, and include amino acid substitutions in which one amino acid having certain physical and/or chemical properties is exchanged for another amino acid that has the same or similar chemical or physical properties. For instance, the conservative amino acid substitution can be an acidic/negatively charged polar amino acid substituted for another acidic/negatively charged polar amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Cys, Val, etc.), a basic/positively charged polar amino acid substituted for another basic/positively charged polar amino acid (e.g. Lys, His, Arg, etc.), an uncharged amino acid with a polar side chain substituted for another uncharged amino acid with a polar side chain (e.g., Asn, Gln, Ser, Thr, Tyr, etc.), an amino acid with a beta-branched side-chain substituted for another amino acid with a beta-branched side-chain (e.g., Ile, Thr, and Val), an amino acid with an aromatic side-chain substituted for another amino acid with an aromatic side chain (e.g., His, Phe, Trp, and Tyr), etc.

The terms “percentage homology” and “percentage sequence identity”, when used in reference to a polypeptide or polynucleotide sequence, are used interchangeably herein to refer to comparisons among polynucleotides and polypeptides, and are determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Homology is evaluated using any of the variety of sequence comparison algorithms and programs known in the art. Such algorithms and programs include, but are by no means limited to, TBLASTN, BLASTP, FASTA, TFASTA, and CLUSTALW [Pearson and Lipman, 1988, Proc. Natl. Acad. Sci. USA 85(8):2444-2448; Altschul et al., 1990, J. Mol. Biol. 215(3):403-410; Thompson et al., 1994, Nucleic Acids Res. 22(2):4673-4680; Higgins et al. 1996, Methods Enzymol. 266:383-402; Altschul et al., 1990, J. Mol. Biol. 215(3):403-410; Altschul et al., 1993, Nature Genetics 3:266-272]. In certain embodiments, protein and nucleic acid sequence homologies are evaluated using the Basic Local Alignment Search Tool (“BLAST”) which is well known in the art (see, e.g., Karlin and Altschul, 1990, Proc. Natl. Acad. Sci. USA 87:2267-2268; Altschul et al., 1990, J. Mol. Biol. 215:403-410; Altschul et al., 1993, Nature Genetics 3:266-272; Altschul et al., 1997, Nuc. Acids Res. 25:3389-3402).

As used herein, the term “isolated” in connection with a compound described herein means the compound is not in a cell or organism and the compound is separated from some or all of the components that typically accompany it in a cell or organism.

As used herein, the term “substantially pure” in connection with a sample of a compound described herein means the sample contains at least 60% by weight of the compound. In certain embodiments, the sample contains at least 70% by weight of the compound; at least 75% by weight of the compound; at least 80% by weight of the compound; at least 85% by weight of the compound; at least 90% by weight of the compound; at least 95% by weight of the compound; or at least 98% by weight of the compound.

As used herein, the terms “treat”, “treating”, “treatment”, and the like refer to reducing or ameliorating a disorder/disease and/or symptoms associated therewith. It will be appreciated, although not precluded, treating a disorder or condition does not require that the disorder, condition, or symptoms associated therewith be completely eliminated. In certain embodiments, treatment includes prevention of a disorder or condition, and/or symptoms associated therewith. The term “prevention” or “prevent” as used herein refers to any action that inhibits or at least delays the development of a disorder, condition, or symptoms associated therewith. Prevention can include primary, secondary and tertiary prevention levels, wherein: a) primary prevention avoids the development of a disease; b) secondary prevention activities are aimed at early disease treatment, thereby increasing opportunities for interventions to prevent progression of the disease and emergence of symptoms; and c) tertiary prevention reduces the negative impact of an already established disease by restoring function and reducing disease-related complications.

As used herein, the term “subject” refers to any animal (e.g., a mammal), including, but not limited to, humans, non-human primates, canines, felines, and rodents.

The comprehensive evaluation of the role of Epigen (also known as epithelial mitogen) in energy metabolism, with a particular focus on glucose homeostasis is described herein. Diet-induced obesity (DIO) and streptozotocin (STZ)-induced diabetic mouse models, along with EPGN KO mice and EPGN overexpressing mice using an adenovirus-mediated gene expression system are utilized. Additionally, the therapeutic potential of Epigen for treating diabetes, with findings that could inform the development of targeted therapies to treat diabetes are investigated.

It will be apparent to those skilled in the art that modifications, including additions and/or substitutions, may be made without departing from the scope and spirit of the invention. Specific details may be omitted so as not to obscure the invention; however, the disclosure is written to enable one skilled in the art to practice the teachings herein without undue experimentation.

A method of treating diabetes or a related complication in a subject in need thereof, the method comprising: administering a therapeutically effective amount of Epigen or a functional fragment thereof to the subject.

The Epigen can be any Epigen known in the art, such as those produced human, bovine, swine, rabbit, rodent, primate, sheep, and goat or a recombinant Epigen produced by, e.g., bacteria.

In certain embodiments, the Epigen comprises or consists of a polypeptide having 77.3% identity and 83.9% similarity at least 95%, at least 96%, at least 97%, at least 98%, at least 99%, or 100% sequence homology with a sequence selected from the group consisting of SEQ ID NO:1 (UniProt ID NO: Q6UW88, human) and SEQ ID NO:2 (UniProt ID NOL: Q924X1, mouse). In certain embodiments, the Epigen is SEQ ID NO:1 or SEQ ID NO: 2.

In certain embodiments, the sequence of the Epigen can differ from SEQ ID NO: 1 or SEQ ID NO: 2 by 1, 2, 3, 4, 5, 6, 7, 8, 9, or 10 amino acid modifications (e.g., insertion, substitution, deletion, etc.). In certain embodiments, the Epigen comprises a polypeptide with conservative amino acid replacements, non-conservative amino acid replacements, or a combination thereof.

Human wild type Epigen can include six cysteine residues, which can form disulfide bonds (e.g., at residues 60-73, 68-84, and 86-95) and two putative N-linked glycosylation sites (e.g., at side chains of asparagine residues 37 and 41). The present disclosure contemplates all such Epigen analogs.

In certain embodiments, the Epigen is a recombinant Epigen, e.g., prepared using a bacterial expression system.

In many bacterial expression systems, the start codon typically codes for methionine, which consequently produces proteins initiated with a N-terminal methionine in these expression systems. However, it is well known that certain bacterial enzymes, such as methionine aminopeptidase (MetAP) and the like, can catalyze the hydrolytic cleavage of the N-terminal methionine from newly synthesized polypeptides. This is commonly observed in instances in which the next amino acid is, e.g., Gly, Ala, Ser, or Thr [In vivo processing of N-terminal methionine in E. coli, FEBS Lett. 1990 Jun. 18; 266(1-2):1-3]. Accordingly, in certain embodiments the Epigen include variants in which the N-terminal methionine of the protein is not present.

In certain embodiments, the Epigen is isolated and/or substantially pure.

It is possible to enhance the biological half-life or bioavailability of the Epigen thereby increasing the retention or stability of the Epigen in a subject, such as by fusion of the Epigen or functional fragment and/or variant thereof with human serum albumin, an albumin binding domain, an Fc region of immunoglobulin, a polyethylene glycol group (PEG) group, or a combination thereof.

The Epigen can be engineered to include specific sites on the protein where PEG can be selectively attached. The selected PEGylation sites are preferably located at a site removed from the binding site of Epigen, and generally exposed to solvent to allow reaction with PEGylation reagents.

The diabetes can be type I diabetes, type II diabetes, type I pre-diabetes, or type II pre-diabetes. The related complication can be any complication of diabetes, such as glucose intolerance, hyperglycemia, insulin resistance, reducing fat mass, pancreatic beta cell dysfunction, hypertension, cardiovascular diseases comprising hypertension, heart attack, and stroke, diabetic nephropathy, diabetic retinopathy, diabetic vasculopathy, diabetic neuropathy, blindness, kidney failure, and lower limb amputation and combinations thereof.

The mode of administration for the Epigen may be any suitable route that delivers the agent to the host, such as parenteral administration, e.g., intradermal, intramuscular, intraperitoneal, intravenous or subcutaneous, pulmonary; transmucosal (oral, intranasal, intravaginal, rectal); using a formulation in a tablet, capsule, solution, suspension, powder, gel, particle; and contained in a syringe, an implanted device, osmotic pump, cartridge, micropump; or other means appreciated by the skilled artisan, as well known in the art. Site specific administration may be achieved by for example intrarticular, intrabronchial, intraabdominal, intracapsular, intracartilaginous, intracavitary, intracelial, intracerebellar, intracerebroventricular, intracolic, intracervical, intragastric, intrahepatic, intracardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravascular, intravesical, intralesional, vaginal, rectal, buccal, sublingual, intranasal, or transdermal delivery.

Alternatively, the Epigen or its derivatives may also be delivered to said subject with an expression vector including an adenovirus-mediated expression vector containing an encoding sequence of the protein or its derivatives to be expressed in host cells or target tissues of the subject.

In certain embodiments, the composition comprising the Epigen may be administered intravenously, intramuscularly, subcutaneously or orally at dosages in a range from about 1 μg/kg to 10 mg/kg body weight, or at a concentration sufficient to produce serum levels of 10⁻¹⁰ M to 10⁻¹¹ M, although a lower or higher dosage may be administered. Dosage is variable subject to various factors including, but not limited to, the severity of the condition of the subject in need thereof, for example, the severity of a patient's hyperglycemia, and upon such criteria as the patient's height, weight, sex, age, and medical history. The dose may also vary depending upon whether the composition of the invention is administered in what setting, e.g., a veterinary setting to a smaller animal or in a physician setting to a human subject.

For the purpose of parenteral administration, the composition comprising the Epigen or its derivatives are preferably dissolved in distilled water and the pH-value is preferably adjusted to about 6 to 8. In certain embodiments, the composition is formulated in lyophilized form. In order to facilitate the lyophilization process, lactose may be added to the solution. Preferably, the solution is then filtered, sterilized, introduced into vials, and lyophilized. In a preferred embodiment, the composition is administered orally to a subject at the time of eating or shortly thereafter. The concentration of the Epigen derivatives in these compositions, and especially the concentration of Epigen whether oral or parenteral, may vary from 10⁻¹² M to 10⁻³ M.

Pharmaceutical compositions of the present invention suitable for parenteral administration comprise the glucoregulatory compound described herein in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain sugars (such as sucrose), alcohols, non-ionic surfactants (such as Tween 20), antioxidants, buffers, bacteriostats, chelating agents, solutes which render the formulation isotonic with the blood of the intended recipient or suspending or thickening agents.

Additional pharmaceutical methods may be employed to control the duration of action. Epigen may be formulated into a controlled release preparation which can be achieved by using certain polymers to complex or adsorb the Epigen or its derivatives. The controlled release system may be enabled by selecting appropriate macromolecules (for example, polyesters, polyamino acids, polyvinyl pyrrolidone, ethylenevinylacetate, methylcellulose, carboxymethylcellulose, and protamine sulfate), the concentration of macromolecules, as well as the methods of incorporation in order to exert control release effect. Another possible method to control the duration of action of the composition is to incorporate the Epigen or its derivatives into certain particles made of a copolymeric material such as polyethylene vinylacetate copolymers. Alternatively, instead of incorporating the Epigen or its derivatives into the copolymeric particles, it is possible to entrap which in microcapsules which are prepared, for example, by coacervation techniques, by interfacial polymerization with hydroxymethylcellulose or gelatin microcapsules and poly (methylmethacrylate) microcapsules, respectively, or in colloidal drug delivery systems, for example, liposomes, albumin microspheres, microemulsions, nanoparticles, and nanocapsules or in macroemulsions. Such teachings are disclosed in Remington's Pharmaceutical Sciences (1980), which is incorporated herein by reference.

To determine functional attributes and efficacy of Epigen and its derivatives, various assays are employed to test the present protein and its derivatives in different in vitro and in vivo models including glucose uptake by adipocytes or insulin secretion measurement from various pancreatic cells/tissues, physiological change and glucose/insulin/pyruvate tolerance tests in high fat diet-induced obese mouse model, streptozotocin-induced diabetic mouse model, etc.

The following examples accompanied with corresponding drawings are intended to better illustrate various embodiments of the present invention. Scope of the present invention should be defined in the appended claims.

### Circulating Epigen is Tightly Regulated by Nutrient Availability

First, we initially examined the tissue expression of EPGN across various tissues. EPGN mRNA was mainly detected at the tongue, stomach, and muscle, with minimal expression in the other tissues examined (CT>35) via RT-qPCR (FIG. 2A). Consistent with the mRNA expression pattern, Epigen was also detected in these tissues where EPGN mRNA levels were high (FIG. 2B). Notably, high levels of Epigen were observed in the liver and kidney through Western blotting (FIG. 2B). As Epigen functions as a hormone, it may be transported from the site of expression to other tissues via circulation. To investigate this further, we measured circulating Epigen levels in mice at different feeding stages, comparing standard chow (STC) and high-fat diet (HFD) conditions (FIG. 2C). Interestingly, under fed conditions, circulating Epigen levels were higher in STC-fed mice compared to their HFD-fed littermates. Additionally, circulating Epigen levels in STC-fed mice varied in response to the fed-fast cycle (FIG. 2D). Specifically, during fasting, circulating Epigen levels in STC-fed mice decreased by 50%, a change not observed in HFD-fed mice (FIG. 2C). Collectively, these findings suggest that circulating Epigen levels are tightly regulated by nutritional status.

### Knockout of EPGN Enhances Metabolic Dysregulation Induced by HFD Treatment

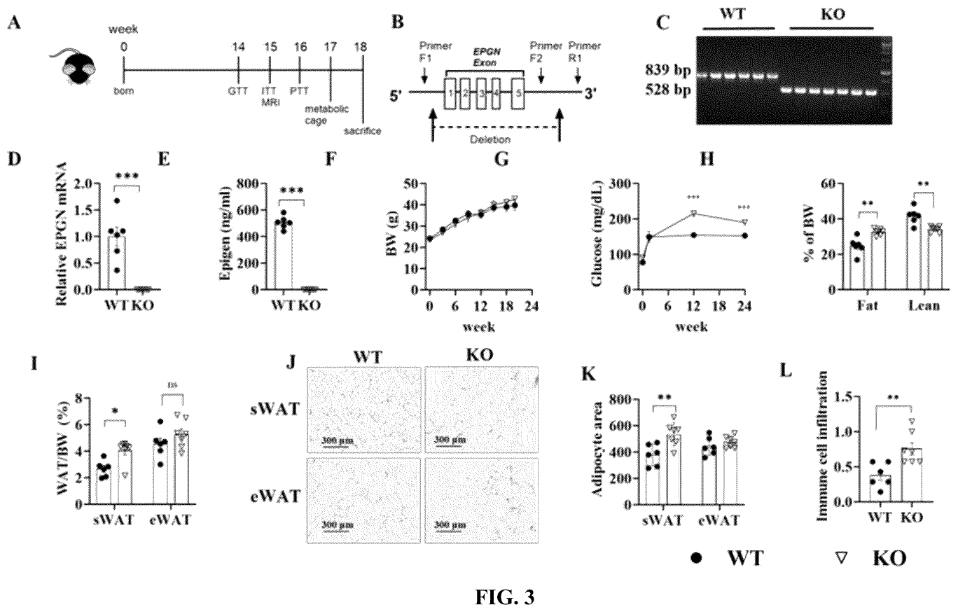

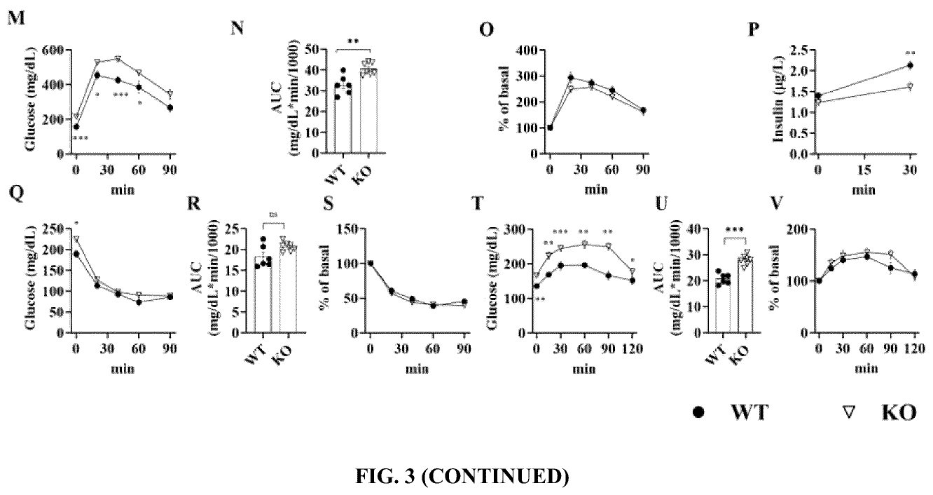

Given the change in the circulating levels of Epigen by nutritional status (FIG. 2) and its family members in energy metabolism, we hypothesized that increased circulating Epigen levels may play a role in combating the pathogenesis of obesity. To assess the importance of Epigen in metabolism, we conducted comprehensive metabolic phenotyping of EPGN knockout (KO) mice (FIG. 3A). The validation of EPGN KO mice was confirmed through genotyping via PCR (FIG. 3B-C), measurement of EPGN mRNA expression by RT-qPCR (FIG. 3D), and assessment of circulating Epigen levels by ELISA (FIG. 3E).

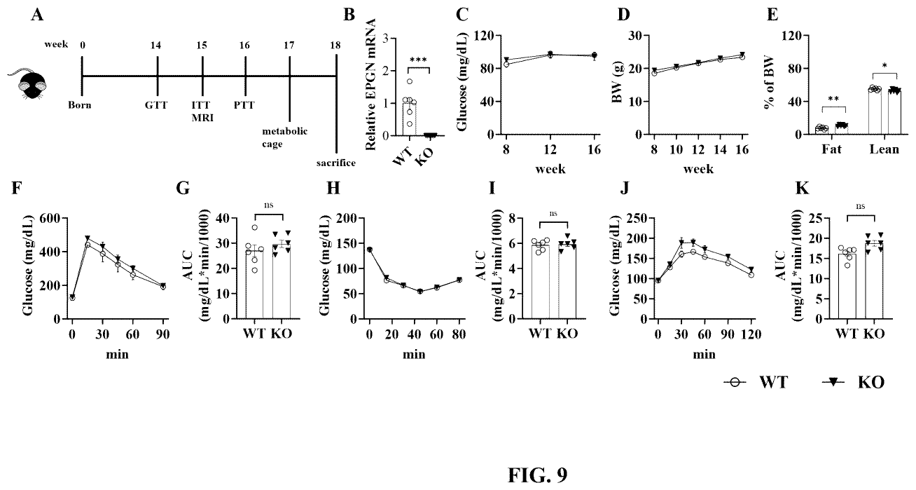

Under standard chow (STC) conditions, EPGN KO mice did not exhibit any obvious metabolic phenotypes (FIG. 9). Although, when subjected to high-fat diet (HFD) conditions (FIG. 3), no statistically significant difference in body weight was observed between wild-type (WT) and EPGN KO mice (FIG. 3F). Notably, EPGN KO mice displayed higher fasting glucose levels (FIG. 3G) and increased fat mass (FIG. 3H). The increase in fat mass was primarily attributed to an increase in the mass (FIG. 3I) and size of adipocytes in subcutaneous white adipose tissue (sWAT) and epididymal white adipose tissue (eWAT) (FIG. 3J-K).

### Impaired Glucose Homeostasis in HFD-Fed EPGN KO Mice

Further impairment in glucose tolerance was observed in HFD-fed EPGN KO mice, as indicated by the glucose tolerance test (GTT) (FIG. 3M-0). This may be attributed to impaired insulin secretion in HFD-fed EPGN KO mice (FIG. 3P). However, the insulin tolerance test (ITT) demonstrated that glucose levels in EPGN KO mice after insulin injection were similar to those of the control group (FIG. 3Q-S). Additionally, EPGN knockout significantly increased hepatic glucose production, as evidenced by sodium pyruvate administration in the pyruvate tolerance test (PTT) (FIG. 3T-V). Taken together, these findings indicate that EPGN knockout worsens glucose intolerance, likely due to impaired insulin secretion, and dysregulation in gluconeogenesis in HFD-fed mice.

### Impaired Lipid Homeostasis in HFD-Fed EPGN KO Mice

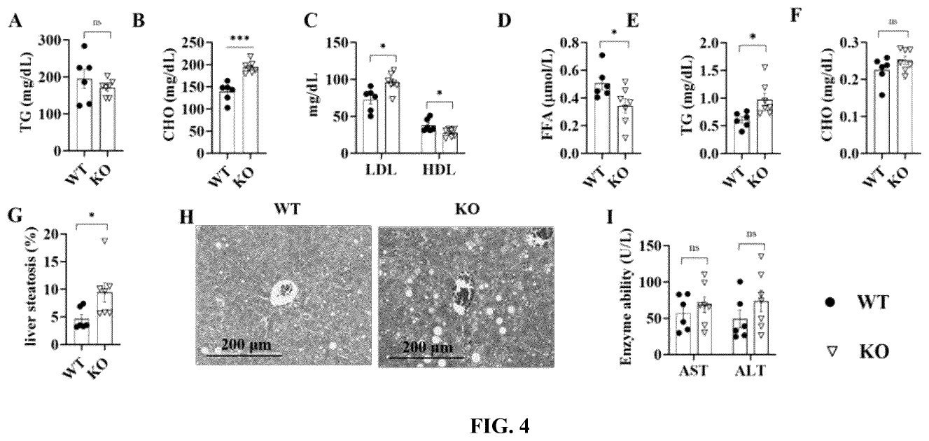

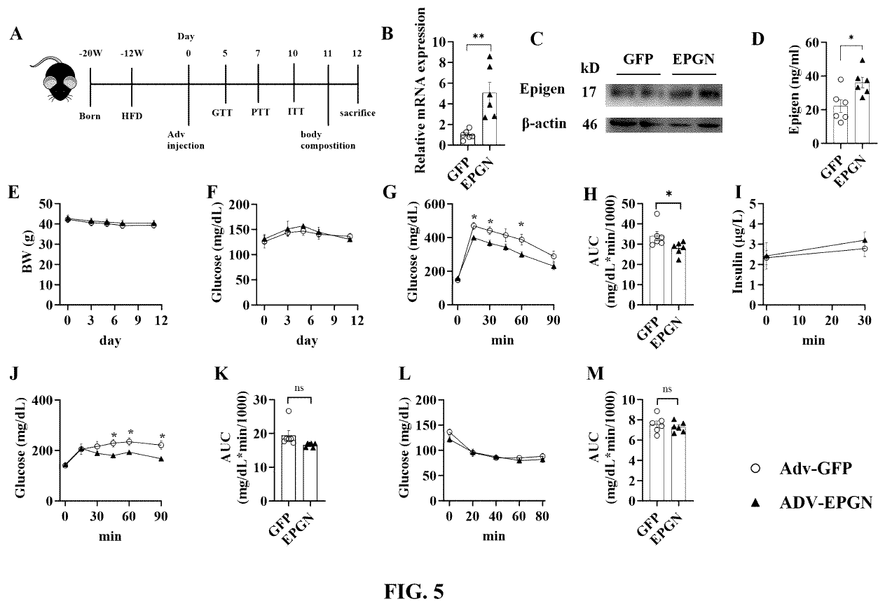

We also examined the lipid profiles of EPGN KO mice. Although there was no significant difference in circulating triglyceride (TG) levels (FIG. 4A), EPGN KO mice exhibited higher circulating cholesterol (CHO) levels compared to their WT littermates (FIG. 4B). The excess cholesterol in the blood was primarily in the form of LDL rather than HDL (FIG. 4C). Interestingly, EPGN KO mice had lower circulating free fatty acid (FFA) levels than their WT counterparts (FIG. 4D). In contrast, EPGN KO mice had higher hepatic triglyceride (TG) levels, but similar cholesterol (CHO) levels compared to their controls (FIG. 4E-F). Lipid accumulation in the livers of EPGN KO mice was greater than in WT mice (FIG. 4G-H). Although EPGN KO mice had increased hepatic lipid levels, their circulating liver damage markers, AST and ALT, were similar to those of WT mice (FIG. 3I).

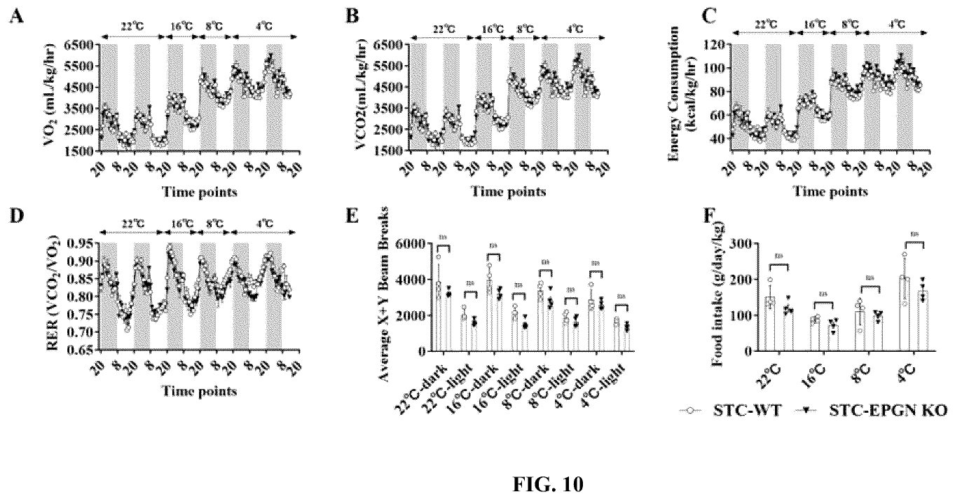

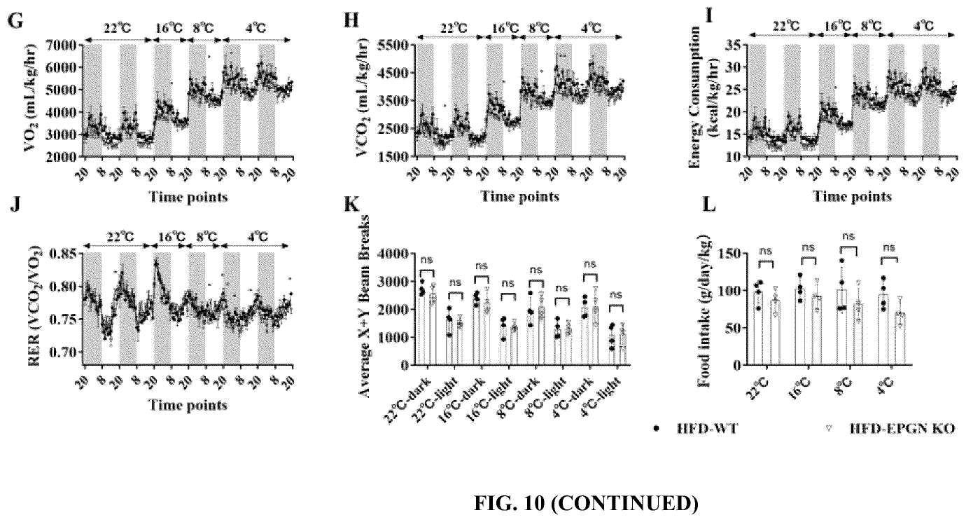

We also used metabolic cages to assess energy expenditure and food intake at various environmental temperatures. Although, there were no differences in VO₂ consumption (FIG. 10A), VCO₂ production (FIG. 10B), energy consumption (FIG. 10C), respiratory exchange ratio (RER) (FIG. 10D), locomotor activity (FIG. 10E), and food intake (FIG. 10F) for most time points between WT and EPGN KO mice, there was a trend indicating that EPGN KO mice had lower VO₂ consumption (FIG. 10A), VCO₂ production (FIG. 10B), energy consumption (FIG. 10C), respiratory exchange ratio (RER) (FIG. 10D) for several data points. Taken together, these findings suggest that the observed differences in EPGN KO mice may be due to intrinsic changes in lipid metabolism, potentially linked to lower energy expenditure.

### Overexpression of EPGN Alleviates Metabolic Dysregulation Induced by HFD Treatment

As EPGN knockout enhances dysregulation of glucose homeostasis (FIG. 3), we further evaluated the impact of Epigen on metabolism by overexpressing EPGN in diet-induced obese (DIO) mice using an adenovirus-mediated EPGN expression system (FIG. 5A). The overexpression was validated by measuring EPGN mRNA levels in the liver via RT-qPCR (FIG. 5B), hepatic Epigen levels through Western blotting (FIG. 5C), and circulating Epigen levels by ELISA (FIG. 5D). Overexpression of EPGN did not affect body weight (FIG. 5E) or fasting glucose levels (FIG. 5F). However, improvements in glucose and pyruvate tolerance were observed, as indicated by the glucose tolerance test (GTT) (FIG. 5G-H) and pyruvate tolerance test (PTT) (FIG. 5J-K). There was no change in insulin sensitivity, as demonstrated by the insulin tolerance test (ITT) (FIG. 5L-M). Taken together, these results indicate that overexpression of EPGN can improve glucose and pyruvate tolerance in diet-induced obese mice without affecting body weight or insulin sensitivity.

### Acute Injection of High Dosage Epigen Improves Glucose Homeostasis

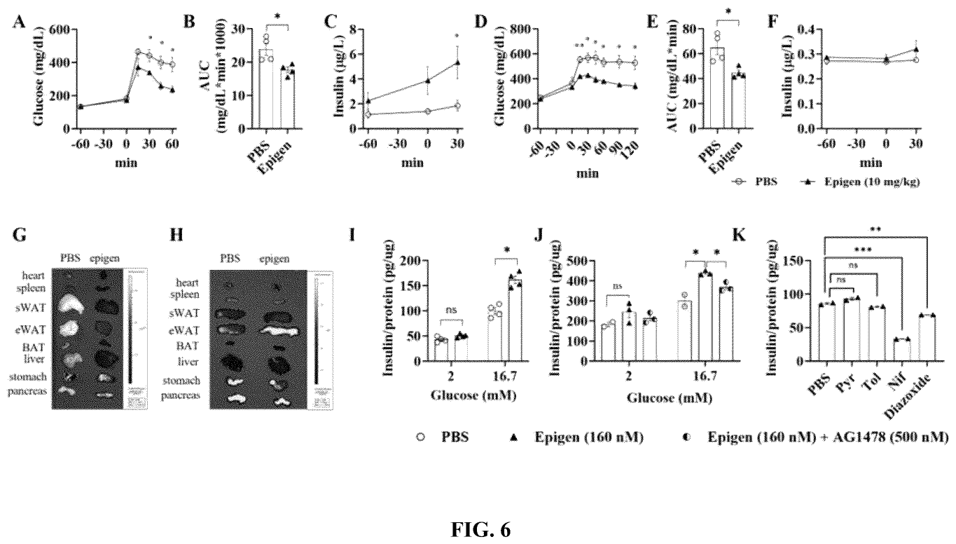

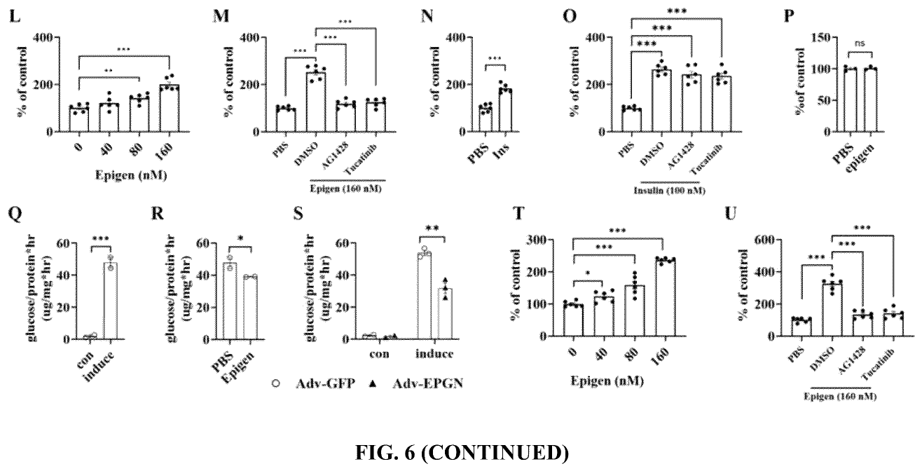

As Epigen is hormone, we explored whether injection of Epigen has similar benefit effect of overexpressing EPGN in mice. In brief, intraperitoneal injection of 10 mg Epigen/kg into HFD-fed mice can enhance the glucose tolerance as demonstration with GTT as compared to control group injected with PBS (FIG. 6A-B). Injection of Epigen significantly increased the circulating insulin level (FIG. 6C). Surprisingly, injection of Epigen also could enhance glucose tolerance in STZ-treated mice (FIG. 6D-E) without altering circulating insulin level (FIG. 6F).

Epigen Induces Insulin Secretion from Pancreatic Beta Cells

To determine the target organ through which Epigen regulates glucose homeostasis, we utilized fluorescent labeling of Epigen along with a fluorescent glucose tracer, 2-N-7-nitrobenz-2-oxa-1,3-diazol-4-ylamino-2-deoxyglucose (2-NBDG), followed by imaging with an in vivo fluorescence imaging system (IVIS) Spectrum system. The labeled Epigen accumulated in all examined organs (FIG. 6G). Intriguingly, treatment with exogenous recombinant Epigen promoted the uptake of 2-NBDG by eWAT (FIG. 6H).

As acute injection of Epigen increased circulating insulin level in HFD mice (FIG. 6A-C) Significant amount of labeled Epigen accumulated at pancreas, we explored whether Epigen can induce insulin secretion from pancreatic beta cells by in vitro experiments. Epigen could induced insulin secretion from SJ β cells in dosage dependent manner at high (16.7 mM) glucose level but no at low (2 mM) glucose level (FIG. 6I), and the induction could be blocked by EGFR inhibitor AG1478 (FIG. 6J), and calcium channel inhibitor Nifedipine (Nif; FIG. 6K).

### Epigen Promotes Glucose Uptake by Adipocytes and Muscle Cells, and Represses Glucose Production by Hepatocytes

To further investigate whether Epigen can directly enhance glucose uptake in adipose tissues, we conducted in vitro glucose uptake assays using differentiated 3T3-L1 cells. Treatment with recombinant Epigen enhanced glucose uptake by differentiated 3T3-L1 cells in a dose-dependent manner (FIG. 6L), and this effect could be blocked by EGFR inhibitors, AG1478 and tucatinib (FIG. 6M). Insulin was used as a positive control for glucose uptake in 3T3-L1 cells (FIG. 6N). Additionally, consistent with the IVIS data for the accumulation of 2-NBDG in eWAT (FIG. 6H), treatment with Epigen did not further increase glucose uptake in the liver cell line HepG2 (FIG. 6P).

Although Epigen did not promote glucose uptake in the liver, the findings that EPGN knockout worsened glucose metabolism while EPGN overexpression and Epigen treatment enhanced pyruvate tolerance (as demonstrated by the pyruvate tolerance test, PTT) prompted us to explore whether Epigen can inhibit liver utilization of pyruvate by repressing gluconeogenesis. Treatment of primary mouse hepatocytes with glucagon and pyruvate increased glucose levels in the cells via gluconeogenesis (FIG. 6Q). However, treatment with recombinant Epigen (FIG. 6R) or overexpression of EPGN (FIG. 6S) in primary hepatocytes significantly lowered glucose levels.

Given that muscle tissue is generally recognized as a key organ for glucose uptake GTT, we also examined whether recombinant Epigen could enhance glucose uptake in muscle cells. Notably, Epigen also increased glucose uptake by differentiated L6 muscle cells in a dose-dependent manner (FIG. 6T), and this effect could similarly be blocked by EGFR inhibitors (FIG. 6U).

### Chronic Injection of Low Dosage Epigen Also Alleviates Metabolic Dysregulation Induced by HFD Treatment

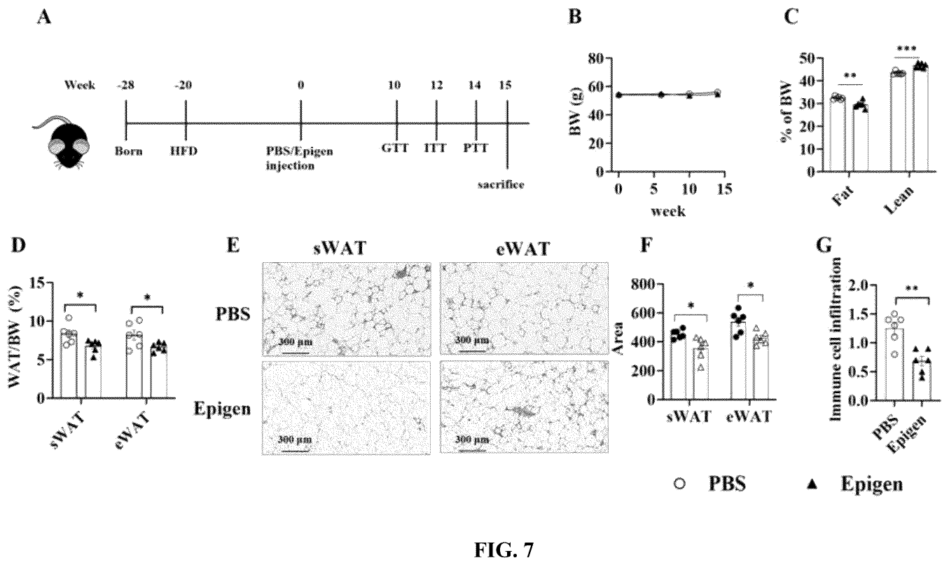

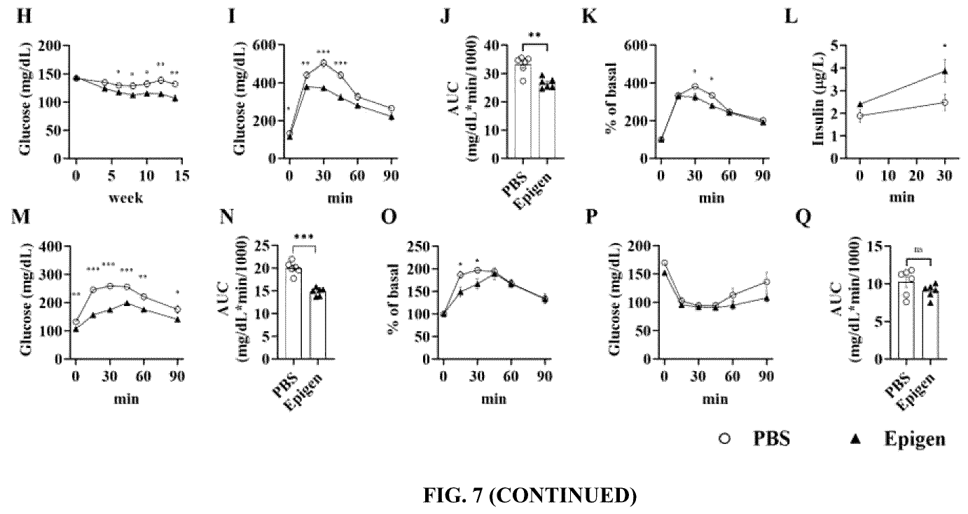

To explore the pharmaceutical potential of long-term Epigen injection, we administered a low concentration of recombinant Epigen (2 mg/kg) for 15 weeks (FIG. 7A). Although chronic injection of Epigen did not affect body weight throughout the study (FIG. 7B), it significantly reduced fat mass and increased lean mass (FIG. 7C). The weight of both subcutaneous white adipose tissue (sWAT) and epididymal white adipose tissue (eWAT) in the Epigen-injected groups were lower than those in the PBS controls, likely due to the smaller size of adipocytes in both sWAT and eWAT (FIG. 7D-F). Importantly, fasting glucose levels in the Epigen treatment group were lower than those in the control group starting at week 6 (FIG. 7H). Chronic injection of low-dose Epigen also improved glucose and pyruvate tolerances, as demonstrated by the GTT (FIG. 7I-L) and PTT (FIG. 7M-O), respectively, but did not enhance insulin sensitivity (FIG. 7P-Q).

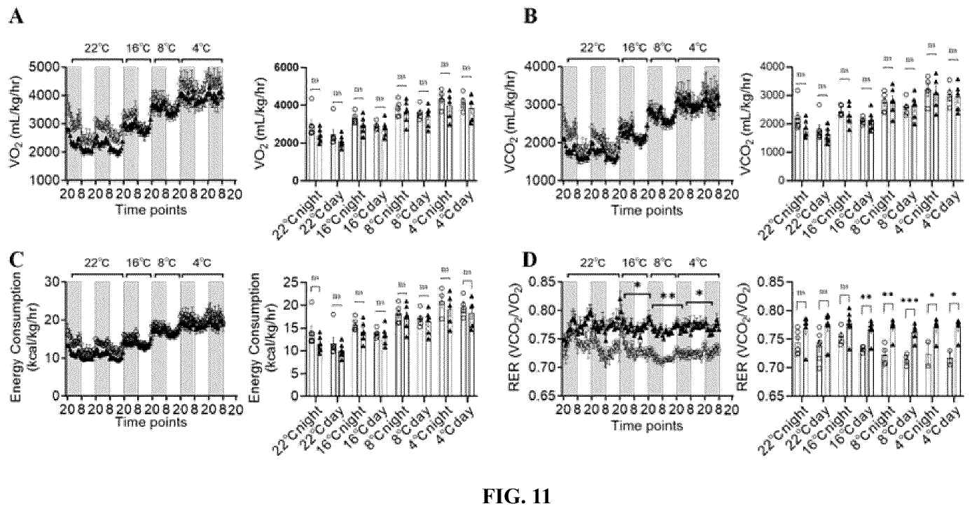

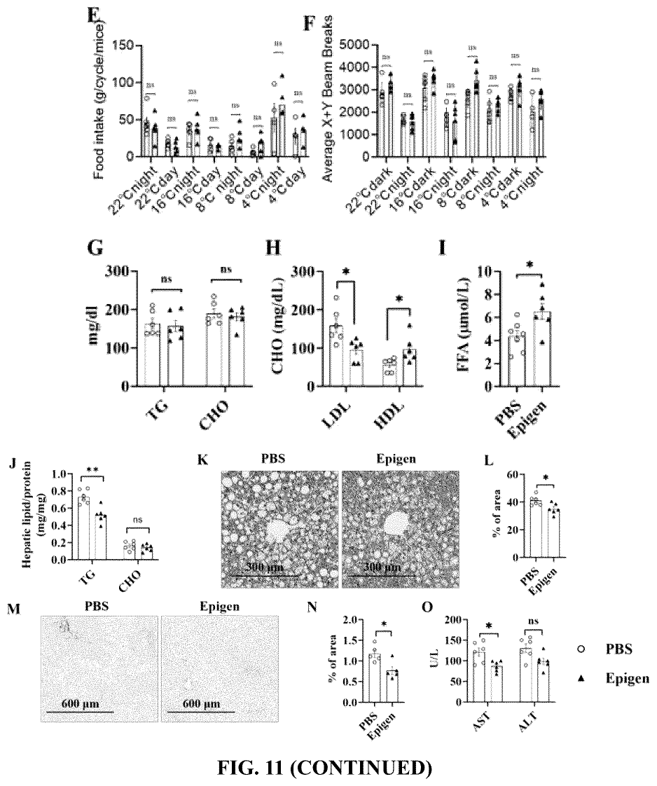

Although there were no differences in VO₂ consumption (FIG. 11A), VCO₂ production (FIG. 11B), energy consumption (FIG. 11C), locomotor activity (FIG. 11E), and food intake (FIG. 11F) between the groups, chronic injection of Epigen significantly increased the respiratory exchange ratio (RER), particularly under lower temperature conditions (FIG. 11D). While there were no differences in circulating triglycerides (TG) and cholesterol (CHO) levels (FIG. 11G), chronic injection of low-dose Epigen lowered LDL levels and increased HDL levels (FIG. 11H), as well as increased circulating free fatty acids (FFA) (FIG. 11I) in HFD-fed mice. Hepatic TG level was decreased and no change in hepatic CHO level (FIG. 11J). In agreement with lower hepatic TG level, lipid accumulation in the livers of mice receiving chronic Epigen injection was lower than in the PBS control group, as demonstrated by H&E staining (FIG. 11K-L). Less fibrosis and liver damage were also observed in the chronic Epigen injection group, as shown by Sirius Red staining (FIG. 11M-N) and biochemical assays (FIG. 11O). Taken together, these findings suggest that long-term injection of Epigen may have beneficial effects on body composition, glucose metabolism, and liver health in diet-induced obese mice.

### Epigen Promotes Pancreatic Beta Cell Regeneration

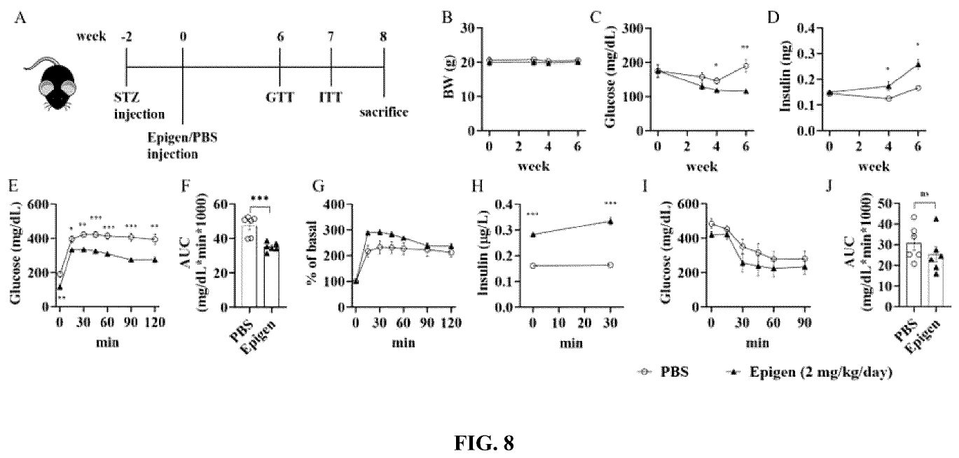

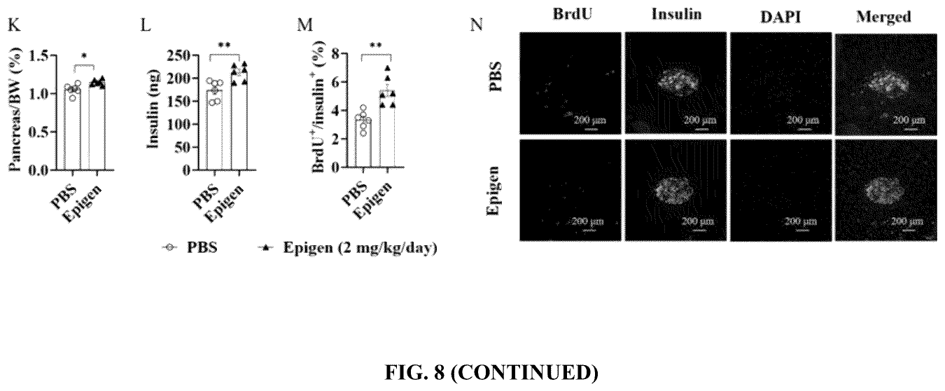

As previous studies have shown that various EGFR ligands can promote pancreatic beta cell regeneration, we investigated whether Epigen could also facilitate this process. We administered a low dosage (2 mg/kg) of Epigen via IP injection to STZ-induced diabetic mice for a duration of 8 weeks (FIG. 8A). Chronic administration of Epigen did not significantly impact the body weight of the STZ-induced diabetic mice (FIG. 8B). However, we observed a reduction in fasting glucose levels starting from week 4 (FIG. 8C) and an increase in endogenous insulin levels beginning at week 6 (FIG. 8D). Consistent with the data obtained from HFD mice in FIG. 7, chronic injection of low-dose Epigen improved glucose tolerance, as evidenced by the GTT results (FIG. 8E-F). Notably, insulin sensitivity was not enhanced, as demonstrated by the ITT results (FIG. 8I-J). Furthermore, treatment with Epigen resulted in a slight increase in pancreatic mass (FIG. 8K) and elevated insulin content within the pancreas (FIG. 8L). The BrdU incorporation into insulin-secreting cells was significantly higher in the Epigen treatment group compared to the PBS control. In summary, our findings suggest that Epigen treatment can promote pancreatic beta cell regeneration (FIG. 8M-N).

The foundation research on EGFR and its role in cellular processes, including its involvement in development and tissue repair. Dysregulation of EGFR signalling has been implicated in various diseases, particularly cancer. Aberrant activation of EGFR can promote tumour growth, invasion, and metastasis. Therefore, EGFR has been an important target for therapeutic interventions in cancer treatment. For example, EGFR inhibitors, including tyrosine kinase inhibitors (TKIs) and monoclonal antibodies, have been developed for clinical use to interfere with EGFR signalling and suppress the growth of cancer cells. However, it is worth noting that some of the EGFR TKIs, like rociletinib, have been associated with dose-limiting hyperglycemia. Patients receiving rociletinib treatment have reported the need for dose reduction, administration of oral antihyperglycemic agents, or both, to manage hyperglycemia while continuing therapy. This suggests that EGFR signalling plays an important role in glucose homeostasis.

Previous studies have attempted to use EGFR ligands for diabetes treatment especially for the survival of pancreatic beta cells. Activation of EGFR has been shown to promote beta cell proliferation, differentiation, and survival, ultimately facilitating insulin production and release. The first in vivo study focusing on the early neonatal period of rats revealed that EGF treatment resulted in elevated plasma insulin concentrations and a higher proportion of beta cells within the islets of Langerhans in 1995. Mechanistically, this improvement can be attributed to the regulation of survivin by EGF through the Raf-1/MEK/ERK pathway in pancreatic J-cells, where EGF prolongs the half-life of the survivin protein and inhibits its ubiquitin-mediated proteasomal degradation. In addition to EGF, other EGFR ligands have also been reported to increase beta cell mass. For example, overexpression of TGFα has been shown to induce the initiation of islet neogenesis via PDX1. In vitro, betacellulin can convert acinar cells into insulin-producing cells; however, in animal models, gene therapy using betacellulin and neurogenin 3 delivered via adenoviral vectors has reversed major metabolic problems in insulin-deficient diabetic mice. Overexpressing HB-EGF also has a significant role in promoting j-cell proliferation via mTOR, MAPK, PI3K/AKT, and IRS2 pathways as demonstrated by transgenic rat model. However, the evidence for epiregulin promotes the proliferation and secretory function limited to rat insulinoma cell lines. While it remains unknown whether amphiregulin can promote j-cell proliferation, it appears to be a common feature among EGFR ligands. Key questions remain regarding which EGFR ligands is most practical, potent, and safe for use in humans. Although gene therapies for treating type 1 diabetes were proposed, many hurdles have prevented their application in humans. In contrast, recombinant protein treatment still represents a more realistic approach. Previous studies have shown that twice-daily intraperitoneal injections of EGF (1 g/kg) and gastrin (3 g/kg) for two weeks restored normoglycemia after diabetes onset in NOD mice, whereas EGF or gastrin alone did not. In this study we showed treatment of recombined Epigen protein (2 mg/kg/day) can increase the endogenous insulin level and pancreatic beta cell mass in STZ-treated mice with 6 weeks. It remains to be explored whether this is a unique feature of Epigen in a specific mouse model or just higher concentrations of EPGR ligands was used to promote j-cell regeneration and proliferation. More comprehensive experiments with detailed comparisons of each EPGR ligand across various preclinical type 1 diabetes mouse models are required.

In addition to demonstrating that recombinant Epigen protein can promote beta cell regeneration and proliferation, we also showed that it enhances insulin secretion via calcium influx. This finding aligns with previous in vivo studies involving recombinant EGF treatment. Specifically, intravenous injection of recombinant EGF stimulates insulin secretion through a concentration-dependent mechanism involving calcium influx and phospholipase D (PLD) activity, which can be inhibited by the EGFR blocker AG1478. Notably, the glucose-lowering effect of intravenous injection of EGF can lower glucose level via insulin. It was reported that 50 g/kg EGF was found to have a potency similar to that of 0.06 U/kg insulin in db/db mice. However, the use of intravenous EGF is not practical for human patients. Such injections are typically limited to ICU settings for selected critically ill patients experiencing diabetes emergencies or other conditions that require rapid control of hyperglycemia. Several laboratories report different effects of epidermal growth factor (EGF) stimulates glycogenolysis in mouse liver and causes mild hyperglycaemia in mice. In contrast to EGF, higher dosages of Epigen administered via intraperitoneal injection resulted in a significant acute reduction in circulating glucose levels, as demonstrated by the GTTs without causing notable hypoglycemia or hyperglycemia in both DIG and STZ diabetic mice.

In addition, without insulin, treatment with Epigen only can enhance glucose uptake in adipocytes and muscle cells through an EGFR-dependent mechanism. We are particularly interested in why Epigen enhances glucose uptake by adipocytes without increasing fat mass, as similar observations have been reported for other EGFR ligands. For examples, systemic administration of EGF reduces fat mass in rats by increasing lipolysis, as indicated by heightened HSL activity and increased HSL mRNA levels; HB-EGF inhibits the early stages of adipocyte differentiation by decreasing the expression levels of adipocyte molecular markers and lipogenic genes; and transgenic mice specifically expressing amphiregulin in white adipose tissue (WAT) also exhibit reduced adipose tissue mass, accompanied by increased mRNA expression levels of TNF-α and peroxisome proliferator-activated receptor γ coactivator 1α (Pgc-1α). Surprisingly, we found that genes and pathways involved in the protein synthesis machinery are downregulated. This observation leads us to hypothesize that there may be a novel pathway through which Epigen limits adipocyte differentiation and function. Further investigation into this mechanism is warranted to clarify the role of Epigen in adipose tissue regulation.

EGFR ligands are proposed to treat various diseases, including metabolic disorders, but their involvement raises significant concerns regarding potential tumorigenesis. Nuclear-localized EGFR is closely associated with disease progression, resulting in worse overall survival in numerous cancers and enhanced resistance to radiation, chemotherapy, and anti-EGFR therapies such as gefitinib and cetuximab. EGF, HB-EGF, and TGF-α are known to facilitate EGFR nuclear translocation, whereas amphiregulin and epiregulin do not promote this process. While EGF, HB-EGF, and TGF-α exhibit high affinity for EGFR, amphiregulin and epiregulin have lower affinities, and the role of Epigen in promoting nuclear localization of EGFR remains to be explored. Previous studies involving EPGN transgenic mice specifically those overexpressing EPGN in the skin observed peripheral demyelination and sebaceous gland hyperplasia. In this study, we did not observe any tumorigenesis in mice injected with adenovirus overexpressing EPGN or through chronic injection of Epigen. However, EGFR ligand can induce tumorigenesis by various mechanisms. Further investigation into the chronic treatment of Epigen is essential, as it may drive aberrant signalling pathways that could contribute to cancer development. Taken together, this study demonstrates Epigen can be a potential target further development for treatment of both type 2 and 1 diabetes.

### EXAMPLES

### Animal Experiments

All animal experiments were approved by Department of Health HKSAR Government (Ordinance Cap. 340), and the Animal Subjects Ethics Sub-Committee (ASESC) of the Hong Kong Polytechnic University. C57BL/6J wild-type mice were housed in the Centralized Animal Facilities of the Hong Kong Polytechnic University at 23° C.±1° C. on standard 12 h light/12 h dark cycle with ad libitum access to drinking water and diet of standard chow (STC) or high fat diet (HFD).

For adenovirus-mediated EPGN overexpression, 10⁹ PFU adenovirus expressing GFP (Adv-GFP) or Epigen (Adv-EPGN) were injected into mice via intravenous (IV) injection.

### Glucose Tolerance Test (GTT), Insulin Tolerance Test (ITT) and Pyruvate Tolerance Test (PTT).

For GTT, mice were fasted for 16 hours on settled duration each time. The fasted mice were intraperitoneally injected with glucose dissolved in sterile PBS as indicated dosage. Blood glucose levels were measured from the tail vein at indicated time points accordingly by glucose meter (Roche Diagnostics).

For ITT, mice were fasted for 4-6 hours on settled duration each time. Insulin powder was dissolved then progressively diluted in sterile PBS for delivery. The fasted mice were intraperitoneally injected with insulin as indicated dosage. Blood glucose levels were measured from the tail vein at indicated time points accordingly by glucose meter (Roche Diagnostics).

For PTT, mice were fasted for 16 hours. Sodium pyruvate was dissolved in sterile PBS for delivery. The fasted mice were intraperitoneally injected with pyruvate solution as indicated dosage. Blood glucose levels were measured from the tail vein at at indicated time points accordingly by glucose meter (Roche Diagnostics).

### Assessment of Indirect Calorimetry Using the Promethion Metabolic System

Promethion metabolic cage system (8 cages, Sable Systems) was used to monitor indirect calorimetry of mice. The mice were housed individually in metabolic cage and fed for 24 hours with free food and water. Oxygen consumption rate (VO₂), carbon oxide production rate (VCO₂), respiratory exchange rate (RER), and energy expenditure (kcal) were recorded at different temperatures. The food, water consumption and body weight were recorded by the system. The horizontal and vertical movement (XYZ-axis) in the metabolic cage were monitored using ER4000 Energizer/Receiver for the quantitative measurement of physical activity of mice. The metabolic and behavioral data was integrated by the MacroInterpreter analysis software. VO₂ and VCO₂ were normalized by body weight.

### Glucose-Stimulated Insulin Secretion (GSIS)

For GSIS in mice, the serum separated from blood collected from tail vein at indicated time points in GTT was subjected to measurement of insulin using a mouse insulin ELISA kit (Mercodia).

SJ β-cells were prepared for analysis by washing in secretion buffer (137 mM NaCl, 0.9 mM CaCl₂, 2.7 mM KCl, 1.5 mM KH₂PO₄, 0.5 mM MgCl₂, 8.1 mM Na₂HPO₄, 20 mM HEPES pH 7.4, 0.2% BSA), incubation in secretion buffer containing 2 mM glucose (30 min) and again washing in secretion buffer. The assay was then performed by incubation for 30 min in secretion buffer containing indicated concentrations of glucose and secretagogues. The insulin level was determined using a mouse insulin ELISA kit (ImmunoDiagnostics).

### Serum Chemicals Test

The blood was collected from heart after sacrificed or tail vein puncture. The blood stayed in room temperature at least for 30 min then centrifuged with 3000 rpm for 15 min at room temperature, the supernatant serum was separated and centrifuged with 8000 rpm for 15 min at 4° C. Serum was stored at −80° C. until further analysis. Blood chemicals or enzymes, such as total triglyceride (TG), total cholesterol (CHO), alanine aminotransferase (ALT), aspartate aminotransferase (AST), low-density-lipoprotein-cholesterol (LDL-C), and high-density-lipoprotein-cholesterol (HDL-C) was measured using assay kits (BioSino).

### Cell Culture

3T3-L1 and L6 cell line were grown and maintained in high-glucose DMEM containing 10% FBS and 1% antibiotic-antimycotic mixture in an atmosphere of 5% CO₂ at 37° C. SJ β-cells, an immortalized pancreatic 3-cell line (Radvanyi et al, 1993), were grown and maintained in same condition with additional 55 M 2-Mercaptoethanol.

### 3T3-L1 Differentiation

3T3-L1 cells were seeded in 12-well plates. 2 days after the cells reach confluency, they were changed to MDI induction medium (high glucose DMEM with 50 mM IBMX, 1 mM dexamethasone, 10 μg/mL insulin) for 3 days. The cells were then incubated with insulin medium (high glucose DMEM with 10 μg/mL insulin) for 3 more days. Finally, the cells were incubated with high glucose DMEM for 4 more days.

### Myotube Differentiation

Myotube cells were differentiated from L6 myoblasts cell line. L6 cells were seeded in 96-well plates until confluency. L6 myoblasts were differentiated by switching myoblast from high glucose DMEM with 10% FBS to high glucose DMEM with 2% horse serum for 4-5 days.

### Glucose Uptake Assay

The differentiated cells were fast in serum-free high glucose DMEM overnight, followed by starvation in glucose-free DMEM for 3 hours. The starved cells were incubated with indicated compound for various time before glucose uptake assay start. The glucose uptake assay was performed using glucose uptake kit followed by manufactory's instructions (Abcam).

### Oil Red O Staining

The cultured cells were fixed in formalin, briefly washed with PBS for 5 min. Rinse with 60% isopropanol. Stain with freshly prepared Oil Red O working solution for 15 min. Rinse with 60% isopropanol. The nuclei were stained with Hematoxylin.

### Primary Hepatocyte Isolation and Glucose Production Assay

Liver was cannulated via inferior vena cava and perfused with a pre-warmed buffer, followed by digestion with buffer containing collagenase Type IV (0.5 mg/mL, Catalog #17104019, Thermo Fisher Scientific). The digested liver was gently dissociated in cold complete William's E medium to stop digestion. The cell suspension was filtered by a 100 m Falcon strainer and washed with cold PBS by centrifugation at 50 g for 2 min at 4° C., followed by resuspension with complete William's E medium.

Hepatocytes with 2*10⁵/well density were seeded in 12 well plates, cultured at 37° C. for 4 hours, then changed with fresh complete William's E medium and incubated for 20 hours. Cells were then washed by Hanks' Balanced Salt Solution (HBSS, 127 mM NaCl, 3.5 mM KCl, 0.44 mM KH₂PO₄, 4.2 mM NaHCO₃, 0.33 mM Na₂HPO₄, 1 mM CaCl₂, 20 mM HEPES, pH 7.4) for 3 times and starvation in HBSS for 2 hours, then washed again. Primary hepatocytes were incubated for 3 hours in HBSS buffer containing indicated reagents. After 3 hours of incubation, the culture medium was collected and centrifuged. The concentration of glucose in the supernatant was measured with the Glucose content assay kit (Catalog #BC2505, Solarbio Science and Technology) according to the manufacturer's instructions.

### Real-Time PCR

Total RNA was extracted from mice tissues with TRIzol and 2.5-5 μg of total RNA was used for cDNA synthesis by reverse transcription reaction. The expression level of target genes was detected using SYBR Green with the specific primers on Real-Time PCR System. Housekeeping genes were used for normalization.

### Immunoblotting

Tissues were homogenized in RIPA lysis buffer and protein concentration was determined by BCA protein quantification assay kit. Extracted proteins were denatured by sample loading buffer under 95° C. for 5 min. Equal proteins were loaded into and separated by sodium dodecyl sulphate-polyacrylamide gel electrophoresis (SDS-PAGE), and then transferred to PVDF membrane with constant 100 voltage for 90 min. After blocking with 5% non-fat milk in 1×TBST for 1 hour at room temperature, the membrane was probed with different primary antibodies in 5% BSA in 1×TBST at 4° C. for overnight. The membrane was washed with 1×TBST 3 times (5 min each) and subsequently incubated with HRP-conjugated secondary antibody prepared in 5% non-fat milk in 1×TBST for 1 hour at room temperature. Afterwards, the membrane was washed with 1×TBST for four times (10 min each). The specific signal of protein was visualized by ECL western blot detection kit and developed. The intensity of protein bands was quantified by the Image J software, relative target proteins expression levels were normalized by indicated internal control.

### Hepatic Lipid

Approximately 25 mg liver tissues were homogenized with 200 μL PBS, 10 μL of the homogenate was subjected to protein quantification, the rest of homogenate was followed by mixing with 5 mL chloroform-methanol mixture (chloroform:methanol, 2:1, v/v) at 4° C. overnight. An aliquot of 1 mL of the mixture was washed with 200 μL of saline, followed by centrifugation at 5000 g for 15 min. the washing procedure was repeated. The chloroform layer was collected and air-dried, and then resuspended in 100% ethanol. The TG levels were measured using kit and normalized with protein concentration of tissue lysates.

### Histology

### Slide Preparation

After mice sacrifice, small pieces of tissue were placed into tissue embedding cassettes and fixed in 10% neutral formalin for 24 hours. Tissues were then processed with Excelsior™ AS Tissue Processor using the following protocol: 30 min in 75% ethanol; 75 min in 95% ethanol×2 times; 75 min in 95% ethanol×3 times; 60 min in xylene×2 times; Overnight paraffin infiltration at 60° C. Tissues were subsequently embedded in freshly melted paraffin and stored until further analysis.

The paraffin-embedded blocks were cut into 5-μm-thick sections, which were deparaffinized and rehydrated by 15 min xylene×2 times, 5 min 100% ethanol×2 times, 1 min 95% ethanol×2 times, 1 min 70% ethanol×once and then rinsed in tap water, then the slides were ready for following staining.

### Hematoxylin and Eosin (H&E) Staining

For H&E staining, the sections were stained with hematoxylin 30 seconds for liver or brown adipose tissue and 3 min for white adipose tissue. Then the sections were stained with eosin for 45 seconds for liver or browning adipose tissue and 10 min for white adipose tissue, followed by rinse in tap water and dehydration (1 min 70% ethanol once, 1 min 95% ethanol for 2 times, 5 min 100% ethanol for 2 times). Finally, the stained sections were immersed in xylene and then mounted with DPX mounting medium.

### Sirius Red Staining

Collagen I and III fibers was stained by Sirius red to evaluate fibrosis stage in tissue. Sections were immersed in Picro Sirius Red solution and stain for 60 min at room temperature, then rinsed quickly in acetic acid solution, then in absolute alcohol, followed by rinse in tap water and dehydration. Finally, the stained sections were mounted with DPX mounting medium.

### Production of the Recombinant Active Form of Epigen

The backbone plasmid pLJSRSF7 was used to construct the expression plasmid pLJSRSF7-EPGN. The coding sequences of the active form of EPGN were amplified by PCR using synthetic oligonucleotides. The fragment containing the last 134 residues of EPGN was cloned using primers Forward primer: 5′-AAGGATCCGGACCACGCGCATTAAGCCG (SEQ ID NO: 3) and Reverse primer: 5′-TACAAGCTTACTGAATTTCGGTGGTGCCG (SEQ ID NO: 4). The PCR product and vector plasmid pLJSRSF7 were digested with restriction enzyme BamHI and HindIII followed by ligation to generate the expression plasmid. The constructed plasmid pLJSRSF7-EPGN was then transformed into BL21. The colony was inoculated at 37° C. until OD600 reaches 0.4-0.6, followed by induction with 100 M isopropyl β-d-1-thiogalactopyranoside for 6 hours. The bacterial cells were collected, and the target protein was purified with immobilized metal affinity chromatography (IMAC).

### RNA-Seq Analysis

Total RNA in fresh mice tissue was extracted using Trizol reagent and cleaned up by RNeasy Mini Kit. The RNA quality was determined using RNA 6000 Pico kit by Agilent 2100 Bioanalyze. The qualified samples were subjected to RNA-Sequencing. The sequencing of all RNA samples was performed using DNBSEQ platform via 100 bp reads from the paired ends. With raw sequencing data, simple quality control metrics were generated using fastQC. The reads were aligned to the mouse genome (Genome assembly: GRCm39) using STAR. Raw reads counts were quantified using the featureCounts function of the Rsubread package. Differential expression genes (DEGs) analysis was performed using DESeq2, which internally corrected for the library size. For further DEGs analysis, DAVID (http://david.ncifcrf.gov), EnrichR (http://amp.pharm.mssm.edu/Enrichr/), and GSEA (www.gsea-msigdb.org/gsea/downloads.jsp) were used to enrich the pathways with regulated genes.

### Total Insulin Content Determination

The pancreas was dissected, weighted and homolyzed in 5 mL of acid-ethanol (2% concentrated HCL in 100% ethanol), supernatant was isolated after Centrifuge at 4° C. 4000 rpm for 5 min. The insulin content was measured in the supernatant with insulin kit (Mercodia).

### Statistical Analysis

The t-test were used for the comparison between the mean values of the studied parameters. The significance was set at the level ≤0.05. Values are shown in means±SEM.

$${}^{*} p < 0.05 ; {}^{**} p < 0.01 ; {}^{** *} p < 0. 1 .$$

### Example 1—Improvement in Glucose Tolerance by Recombinant Epigen in Diet-Induced Diabetic Model

Turning to FIG. 7A, 8-week-old male C57BL/6J mice (n=6) were fed with a high-fat diet (HFD) for 20 weeks to induce a diabetes mellitus type 2 (DM2) model and then treated with 2 mg/kg (per body weight; hereinafter as “BW”) of recombinant mouse Epigen (synthesized and purified from E. coli expressing mouse Epigen represented by SEQ ID NO: 2 (Q924X1); hereinafter as “mEPGN”) once daily via intraperitoneal (i.p.) injection for 15 weeks to evaluate its effects on glucose uptake and tolerance in an obese, diet-induced diabetic model. Body weight (FIG. 7B), fasting blood glucose level (FIG. 7H) were measured at the beginning of the i.p. injection and then once every 2 weeks until the end of the experiment (i.e., week 5, 10, 15). Another group of HFD-fed mice was injected with PBS once daily and measured with the same biometric data as in the Epigen-injected group (treatment group) once every 2 weeks from the beginning of the injection until the end of the experiment.

As seen from FIG. 7B, both treatment and control groups had similar body weight change throughout the whole experiment. The most obvious change was in the fasting glucose level in the treatment group compared with the control group (FIG. 7H), where the blood fasting glucose level was reduced in the treatment group from Week 6.

Glucose tolerance test (GTT) was also performed in both the treatment and control groups at Day 0 and Week 10 of the experiment. The results are shown in FIGS. 7H-Q. As seen from FIGS. 7I-L, the blood glucose level in the treatment group was more significantly reduced compared with the control group over a 90-minute time course at Day 0. At Week 6, the overall blood glucose level was also lower in the treatment group than that of the control group over the 90-minute time course (FIGS. 7H-I).

From the above results, it is suggested that Epigen can improve the glucose tolerance in an obese, diet-induced diabetic model. It is also expected that human Epigen (represented by SEQ ID NO: 1) having a sequence homology of about 78% to that of mouse Epigen (represented by SEQ ID NO: 2) may also have similar beneficial effect on improving glucose tolerance in an obese, diabetic (type 2 diabetes) human subject.

### Example 2—Improvement in Glucose Tolerance by Recombinant Epigen in Insulin-Deficient Diabetic Model

Turning to FIG. 8A, 8-week-old male C57BL/6J mice (n=6) which were fed with standard chow (STC) diet were treated with streptozotocin (STZ) for 5 days and waited for 9 days to induce diabetes. Similar to Example 1, 2 mg/kg (BW) of recombinant mEPGN was administered to the STZ-induced diabetic mice via i.p. injection once daily for 7 weeks (treatment group). In parallel, PBS was injected to STZ-induced diabetic mice for 7 weeks as a control group. Same biometric data as in Example 1 were measured once every 7 days, and the results are shown in FIGS. 8B-J, respectively. As seen from FIGS. 8B, changes in body weight, was similar between the treatment and control groups throughout the experiment. The main difference between the two groups was observed in fasting glucose and blood insulin level (FIG. 8C-D). As seen from FIG. 8D, the blood insulin levels between the two groups were similar from Week 0 to Week 4. Interestingly, staring from Week 4, the blood insulin concentration was increased in the treatment group till the end of the experiment, but the blood insulin level could not be restored to the level measured at Day 0, whereas there was no change in the insulin level in the control group since Week 4.

GTT was also performed at Week 6 in this STZ-induced diabetic model. As seen from FIGS. 8E, the overall blood glucose level in the treatment group throughout the 90-minute time course at Week 6 was lower than that in the control group.

From the above results, it is demonstrated that Epigen can improve glucose tolerance in an insulin-deficient diabetic model. It can also be contemplated that the glucose uptake induced by Epigen is in an insulin-independent manner. Furthermore, it is demonstrated that Epigen can also increase an endogenous insulin level.

Although the invention has been described in terms of certain embodiments, other embodiments apparent to those of ordinary skill in the art are also within the scope of this invention. Accordingly, the scope of the invention is intended to be defined only by the claims which follow.

What is claimed is:

1. A method of treating diabetes or a related complication in a subject in need thereof, the method comprising: administering a therapeutically effective amount of epithelial mitogen (Epigen) or a functional fragment thereof to the subject.

2. The method of claim 1, wherein the diabetes is type I diabetes, type II diabetes, type I pre-diabetes, or type II pre-diabetes.

3. The method of claim 1, wherein the related complication is selected from the group consisting of glucose intolerance, hyperglycemia, insulin resistance, reducing fat mass, pancreatic beta cell dysfunction, hypertension, cardiovascular diseases comprising hypertension, heart attack, and stroke, diabetic nephropathy, diabetic retinopathy, diabetic vasculopathy, diabetic neuropathy, blindness, kidney failure, diabetic foot and lower limb amputation and combinations thereof.

4. The method of claim 1, wherein the Epigen comprises a polypeptide having at least 95% sequence homology with SEQ ID NO: 1 or SEQ ID NO: 2.

5. The method of claim 1, wherein the Epigen comprises a polypeptide having at least 98% sequence homology with SEQ ID NO: SEQ ID NO: 1 or SEQ ID NO: 2.

6. The method of claim 1, wherein the Epigen comprises a polypeptide having at least 99% sequence homology with SEQ ID NO: SEQ ID NO: 1 or SEQ ID NO: 2.

7. The method of claim 1, wherein the Epigen comprises a polypeptide having SEQ ID NO: SEQ ID NO: 1 or SEQ ID NO: 2.

8. The method of claim 1, wherein the Epigen consists of a polypeptide having SEQ ID NO: SEQ ID NO: 1 or SEQ ID NO: 2.

9. The method of claim 1, wherein the Epigen is a recombinant Epigen.

10. The method of claim 1, wherein the Epigen is in isolated form.

11. The method of claim 1, wherein the diabetes is type I diabetes or type II diabetes and the consists of a polypeptide having SEQ ID NO: 1 or SEQ ID NO: 2.

12. The method of claim 1, wherein the Epigen is administered intravenously, intraperitoneally, or intramuscularly, orally, or subcutaneously.

13. The method of claim 1, wherein the subject is a human.


SEQUENCE LISTING (4 sequences)

SEQ ID NO: 1 - protein, 154 aa, Homo sapiens [sequence omitted]

SEQ ID NO: 2 - protein, 152 aa, Mus musculus
MALGVLIAVCLLFKAMKAALSEEAEVIPPSTAQQSNWTFNNTEADYIEEPVALKFSHPCLEDHNSYCINGACAFHHELKQAICRCFTGYTGQRCEHLTLTSYAVDSYEKYIAIGIGVGLLISAFLAVFYCYIRKRCINLKSPYIICSGGSPL

SEQ ID NO: 3 - DNA, 28 bp, Mus musculus
  source 1..28: mol_type = genomic DNA
aaggatccggaccacgcgcattaagccg

SEQ ID NO: 4 - DNA, 29 bp, Mus musculus
  source 1..29: mol_type = genomic DNA
tacaagcttactgaatttcggtggtgccg